(19) United States
(12) Patent Application Publication
(10) Pub. No.: US 2023/0192907 A1
(43) Pub. Date: Jun. 22, 2023

(54) PROCESS FOR THE PREPARATION OF LOW MOLECULAR WEIGHT LINEAR HYALURONIC ACID AND HYALURONIC ACID SO OBTAINED

(71) Applicant: GLYCORES 2000 S.R.L., Milano (MI) (IT)

(72) Inventors: Pasqua ORESTE, Milano (MI) (IT); Giorgio ZOPPETTI, Milano (MI) (IT)

(21) Appl. No.: 17/918,659

(22) PCT Filed: Apr. 16, 2021
(86) PCT No.: PCT/IB2021/053169

§ 371 Date: Oct. 13, 2022

(30) Foreign Application Priority Data
Apr. 17, 2020 (IT) 102020000008299

(51) Int. Cl. C08B 37/08

(52) U.S. Cl. CPC C08B 37/0072

(57) ABSTRACT

The present invention relates to a process for the preparation of linear low molecular weight hyaluronic acid (LMWHA) or one of its salts, obtained by chemical depolymerization in strong acidic conditions and to the low molecular weight hyaluronic acid so obtained.

The present invention relates to a process for the preparation of linear low molecular weight hyaluronic acid (LMWHA) or one of its salts, obtained by chemical depolymerization in strong acidic conditions and to the low molecular weight hyaluronic acid so obtained.

### BACKGROUND OF THE INVENTION

Hyaluronic acid (HA) is a natural linear polysaccharide belonging to the family of glycosaminoglycans, constituted by a disaccharide repetition formed by glucuronic acid β 1-4 linked to N-acetylglucosamine. It is ubiquitously present in the living organisms and in the last years its application in the pharmaceutical and cosmetic fields as a component in several formulations has gained increased attention.

Today it is industrially produced by fermentation and, according to the polymeric chain length, it can be distinguished in high molecular weight HA (HMWHA) and low molecular weight HA (LMWHA), from 3,000 and 250 kDa and below 250 kDa respectively. These two categories have different characteristics and functionalities and are used to reach different targets.

As demonstrated by S.Misra et al., Frontiers of Immunology, 2015,6, Article 201, the chains of LMWHA with molecular weight from 3 to 6.5 kDa induce angiogenesis in the chick corneal assay, and those from 6 and 20 kDa induce inflammation in dendritic cells.

Several hyaluronic acid depolymerization methods are known in the literature to obtain the formation of shorter chain lengths, which can yield oligomers formed by few disaccharide units, cfr. Stern R. et al., Biotechnology Advances, vol. 25, pag. 537-557, 2007. Such methods can be substantially divided in enzymatic and non-enzymatic methods, being the first ones more precise and easily controlled but more labour-intensive. Among the non-enzymatic methods use of ultrasounds, ultraviolet rays, and/or high temperature, γ-irradiation, and chemical reactions as oxidations or acidic or basic hydrolysis.

Among the main limitations to use the acidic or basic hydrolysis there is the poor reaction control possibility, determined also by the fact that it is often associated to a “peeling effect” of the chain, in other word the loss of units from the reducing chain terminal by a β-elimination with the formation of saccharinic acid. In acidic conditions, in particular, the HA seems to undergo a disordered degradation with racemisation and some Authors, such as Stern R. et al. (supra) reported that there is no molecular weight reduction of HA in reaction conditions below pH 2.

EP3608343 describes a method to prepare LMWHA starting from HA by heating (80-90° C.) in acidic conditions (pH 2.5-3.5). The claimed final product, obtained by degradation starting from a HA with the chains mean molecular weight of 500 kDa, has a chain mean molecular weight of 200 kDa; the reaction proceeds for 15-30 minutes and is arbitrarily stopped by neutralization of the solution. however, such process lacks an empirical marker able to define the exact moment when the reaction must be stopped in order to obtain the desired molecular weight.

So, it is evident that the known processes are not able to produce constant and reproducible low molecular weight hyaluronic acids. Furthermore, the known processes are unable to provide hyaluronic acids having a low molecular weight with a molecular weight within a narrow range of molecular weights, i.e. with low polydispersibility, and practically free of degradation by-products, in particular with molecular weight less than 20kDa which, as known, have undesirable pharmacological properties.

Many commercial ophthalmic formulations as eye drops containing HA, exploit the wound healing and moisturising property of HA, and use the high medium molecular weight HA. However, it is precisely this characteristic that imposes a limited concentration, which normally is around 0.1% and only in rare cases reaches 0.3%, within the eye drops due to the double aspect linked, on the one hand not to reach the solubility limit of the polymer and on the other hand do not make the final solution too viscous.

Applicant believes that the use of LMWHA, if available in a restricted range of molecular weight which guarantees consistent performances, will allow to answer to different pharmacological and cosmetic needs.

For example, LMWHAs with a molecular weight of about 100 kDa and low dispersion allow to prepare eye drops containing a high concentration of active ingredient, without negatively influencing the viscosity nor generating problems due to its solubility limits.

Other reduced ranges of LMWH, for example from 190 kDa to 215 kDa, for example about 200 kDa, from 45 kda to 60 kDa, for example about 50 kDa, can also be useful for other applications.

Therefore the availability of a simple method to yield LMWHA with a specific mean molecular weight within a controlled range of molecular weight and without degradation by-products is needed.

### Objects of the Invention

Object of the present invention is to provide a simple and effective process to obtain, starting from high molecular weight hyaluronic acid (HMWHA), LMWHAs from 50 kDa to 230 kDa, with a mean molecular weight within a specific range, in a reproducible and consistent way, and also the LMWHAs obtained with such process.

### BRIEF DESCRIPTION OF THE FIGURES

FIG. 1 shows the graphics of the trend of the viscosity and of the molecular weight during the depolymerisation reaction in 0.1N HCl at 60° C. of Example 1.

FIG. 2 shows an example of standard curve of the HPLC analysis using standards with absolute molecular weights.

FIG. 3 shows the comparison between the retention time of the product of Example 1 and of the standard with 100 kDa molecular weight.

### DESCRIPTION OF THE INVENTION

Subject-matter of the present invention is a process to prepare a linear LMWHA, which comprises depolymerisation of a linear HMWHA, in particular a controlled depolymerisation of HMWHA in specific conditions which allow to obtain and isolate a low molecular weight hyaluronic acid (LMWHA) characterized by restricted molecular weight ranges within the range from 50 kDa to 230 kDa.

Even not indicated “molecular weight” in the present invention means the weight average molecular weight (Mw).

So, according to one of its aspects, subject-matter of the invention is a process for the preparation of linear LMWHA, which shows restricted ranges of molecular weight within the range from 50 kDa to 230, which comprises the depolymerisation of HMWHA in an aqueous medium at pH lower than 2 , advantageously at pH 1-1.5, more preferably at pH about 1, at a concentration from 0.5 to 1% (w/v), at a temperature from 40 to 80° C., preferably from 50° C. to 70° C., preferably around 60° C. The depolymerisation is performed starting from a linear HMWHA having a molecular weight preferably comprised between 1 and 2 MDa.

The aqueous medium at the indicated pHs of the present invention is preferably acidic by addition of a non-oxidizing strong acid, preferably an inorganic acid, more preferably hydrochloric acid. Preferably said acid is added to a solution of HMWHA in water as a diluted acid, for example having an acid concentration 0.05-0.3N, preferably as hydrochloric acid 0.1-0.3 N, preferably 0.1-0.2 N. The depolymerisation can be stopped by neutralizing the solution, for example using a basic solution, for example, but not limited to, NaOH to pH7 and the product can be recovered by the technologies known by the skilled in the art.

Surprisingly and contrary to the data found in the literature, the reaction proceeds optimally at pH below 2, preferably around pH 1.

As known, for a given polymer, the “intrinsic viscosity” is the viscosity of a solution at various concentrations and is related to the molecular weight of the polymer through the well-known Mark-Houwink-Sakurada equation

$$\left(η\right) = {\text{KM}}^{\text{α}}$$

where K and α are empirical constants calculated for a given polymer in a given solvent at a given temperature.

The intrinsic viscosity at certain molecular weights can be measured for a given polymer in a given solvent, for example for hyaluronic acid in water. Contrary to intrinsic viscosity, “dynamic viscosity” is defined as the measurement of the resistance of a fluid (for example of a solution) to flow, at a given concentration, and depends on various factors such as temperature, salt concentration, pH, etc.

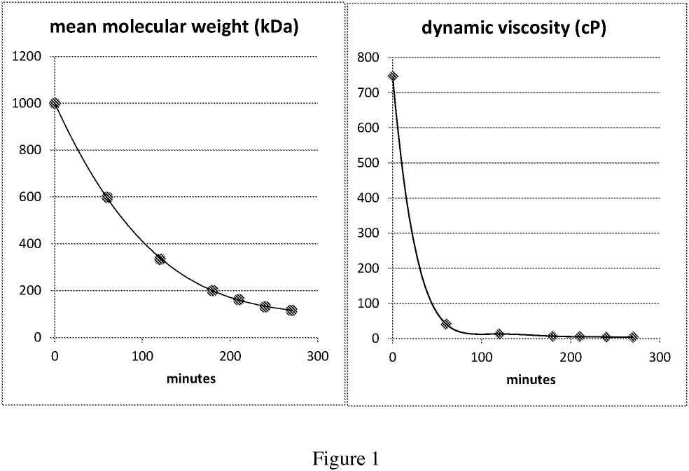

Applicant found that during the depolymerisation reaction of HMWHA, in the conditions of the invention, as the molecular weight of HA descreases, a decrease of the viscosity of the solution occurs following a non-linear kinetic. The viscosity data, actually, after a quick decrease at the initial stage of the depolymerisation reaction, reach a “steady state” in which both molecular weight and viscosity keep on decreasing but with a lower rate (see FIG. 1 referring to Example 1). As can be noticed in FIG. 1, this steady state results in a plateau of the decrease of the viscosity curve relates to the molecular weights from about 50 to about 230 kDa.

Since in the process of the invention during the depolymerization the temperature, the concentration of the acid and the concentration of the product are substantially always the same, or vary within very narrow ranges, the Applicant expected a constant rate of decrease of the molecular weight, also taking into account the fact that the depolymerization occurs in a statistical manner.

Contrary to expectations this did not happen, as shown in FIG. 1.

While not wanting to be bound to any theory, it is assumed that this unexpected phenomenon is due to the rheological characteristics of hyaluronic acid.

In light of this unexpected phenomenon, Applicant decided to exploit the plateau of the curve which, under the conditions of the process of the invention, occurs around molecular weights from about 50 to about 230 kDa, in order to reproducibly obtain LMWHAs having specific and narrow molecular weight ranges.

Applicant, in order to obtain a product with a specific mean molecular weight within a certain and reduced range, thus with a low dispersion, thought to perform the depolymerization of the invention, measuring the viscosity at subsequent times and stopping the depolymerization when the viscosity of the sample reaches values comprised in a specific viscosity range, directly correlated to the values of the polymeric chain length of the desired molecular weight. Such values can be easily predetermined through the evaluation of the decrease of the molecular weight in function of the viscosity during the depolymerisation in the conditions of the process of the invention, building a viscosity curve (FIG. 1).

Therefore, the observation of this unexpected decrease in the depolymerization rate was very useful as, considering the close relationship between the viscosity of the solution and the average molecular weight of the HA chains, it allows to obtain a greater control of the final molecular weight and a consequent better reproducibility of the process.

Moreover it was noticed that by operating at not too much high temperatures, less than 80° C., preferably about 60° C., allows to avoid the formation of degradation sub-products during the reaction.

As already indicated, at the end of the depolymerisation the reaction can be stopped by neutralizing the solution, for example by using a basic solution, such as, but not only, NaOH to a pH of about 7, and the product can be isolated and recovered according to technologies known by the skilled in the art.

In a preferred aspect of the invention, the product can be separated from the solvent by filtration, performed for example but without limiting it, using a filtration membrane for example with 10 kDa cut-off and then precipitating the polymer with a suitable solvent, preferably ethanol, acetone and/or isopropanol. The final product can be obtained by drying, for example maintaining the product in an oven for example at 40° C. for the needed time, for example 4-5 hours or more according to the amount of product prepared.

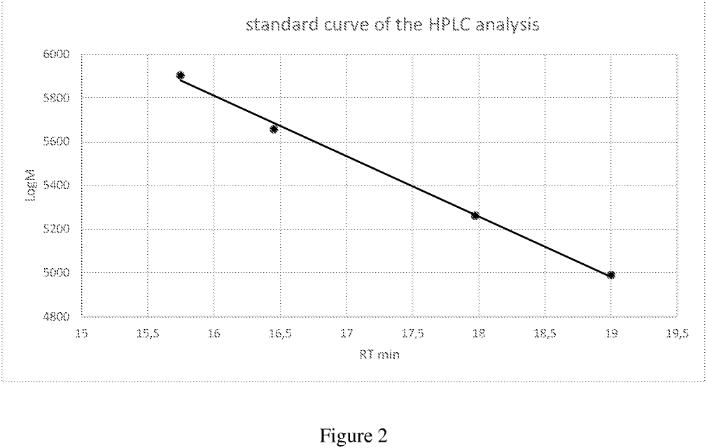

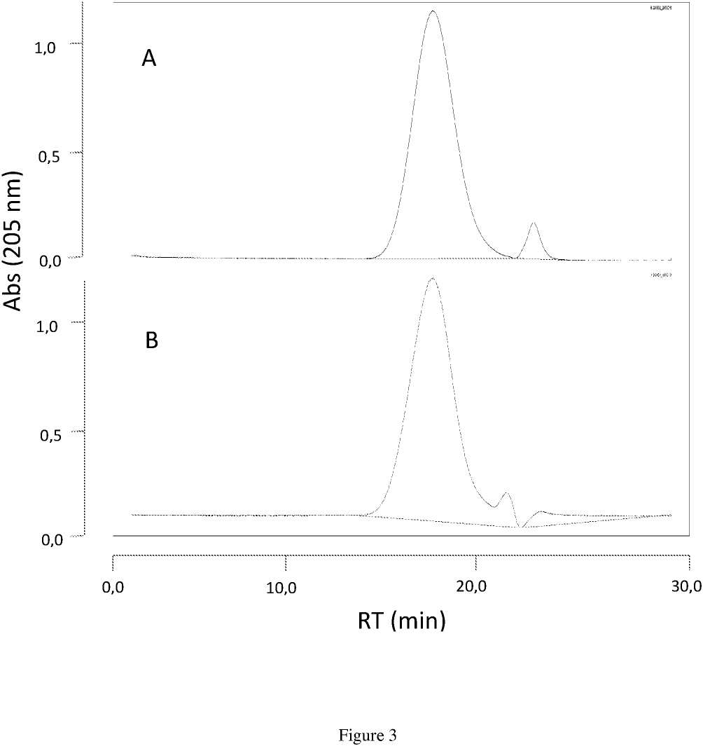

The molecular weight of the LMWHA produced according to the process of the invention can be experimentally measured according to all the techniques known by the skilled in the art: for example by a HPLC method using HA standards with different length of the chains, characterized by a specific absolute mean molecular weight. Such a HPLC method can be preferably coupled to different methods to reveal of the peak elution, in particular viscosity, IR and/o UV. According to a particular aspect of the invention, the peak generated by the sample under examination is compared with the curve obtained with the different standard HA characterized by a specific absolute mean molecular weight to evaluate its molecular weight (see FIGS. 2 and 3 related to Example 1).

With the term “viscosity”, dynamic viscosity is here indicated. Such viscosity is here determined in the analytical conditions described in following the experimental section at about 20° C.

According to an embodiment, the process of the invention comprises the experimental predetermination of a curve which correlates the reaction medium and consequently the molecular weight of the hyaluronic acid therein contained, at certain reaction times, following the process of the invention as defined above, wherein “reaction medium” means the reaction medium after stopping the reaction by the neutralization with a base. This curve can be built operating the process as indicated above and measuring the viscosity at time ranges. By putting the viscosity data, the corresponding molecular weights and times of the stopping of the depolymerisation in a table, a practical instrument to simply obtain the needed degree of depolymerisation becomes available. According to an embodiment, subject-matter of the present invention is a process for the preparation of linear LMWHA having narrow ranges range of molecular weight within the range from 50 kDa to 230 kDa, by a depolymerisation which comprises:
(i) experimentally predetermining a curve correlating the viscosity of the reaction medium and the consequent molecular weight of the hyaluronic acid therein contained as above indicated;
(ii) dissolving linear HMWHA in water, at a concentration of about 1-2% (w/v):
(iii) heating the solution and add a strong acid to obtain pH 1-1,5;
(iv) measuring the viscosity of the solution at different times and stopping the depolymerisation by neutralizing with a strong base till pH about 7 once reached the viscosity related to the LMWHA with the desired molecular weight according to the curve of step (i); and
(v) isolating the LMWHA obtained. By “narrow molecular weight ranges” we mean here that these ranges are generally between 40 and 10 kDa, preferably between 30 and 10 kDa, for example between 20 and 10 kDa, such as about 15-10 kDa.

A further subject-matter of the invention is a LMWHA, obtained according the method of the invention showing the following features:
Mean molecular weight ranging:
   from 50 kDa to 230 kDa; or
   from 90 kDa to 120 kDa, preferably about 100 kDa; or
   from 180 kDa to 230 kDa; or
   from 190 kDa to 215 kDa, preferably about 200 kDa; or
   from 45 kDa to 60 kDa; or
   from 47 kDa to 55 kDa, preferably about 50 kDa;
linear chains (not crosslinked);
less than 5% of the total chains with a molecular weight lower than 20 kDa, measured by HPLC in the conditions indicated in the experimental section below;
polydispersion index lower than 4, preferably lower than 3, more preferably lower or about 2.5.

According to an embodiment, subject-matter of the present invention is also a process for the preparation of linear LMWHA with a molecular weight ranging from 90 kDa to 120 kDa, preferably from 95 kDa to 110 kDa, more preferably about 100 kDa by a depolymerisation which comprises:
a) dissolving the linear HMWHA in water at a concentration of about 1-2 %(w/v);
b) heating the solution and to add a strong acid to a pH of 1-1.5;
c) measuring the viscosity of the solution at time ranges;
d) stopping the depolymerisation adding a strong base when the viscosity reaches 4-5 mPa.s
e) isolating the LMWHA so obtained.

Step (a) is preferably performed in water, at room temperature, wherein “room temperature” means a temperature of 20-25° C., under stirring.

In step (b) the solution is heated to 40-80° C., preferably to about 60° C. The strong, non-oxidizing acid is preferably an inorganic acid, advantageously hydrochloric acid. According to a preferred embodiment, the acid is 0.1-0.3 N hydrochloric acid, advantageously 0.2 N. The pH of the solution preferably is about 1.

In step (c) the viscosity is controlled by known methods. Some examples are reported in the experimental section below. The strong base preferably is an alkaline-metal hydroxide, advantageously sodium hydroxide. The base is added until the solution is neutral, around pH 7.

In step (d) the LMWHA is isolated according to conventional techniques. Some examples are reported in the experimental section below. The indicated viscosity is measured in the analytical conditions described in the experimental conditions below. The LMWHA of the invention, obtained according to the above described process, has a mean chain length in a restricted range, so that the mean molecular weight results from 90 kDa and 120 kDa, preferably from 95 kDa to 110 kDa, more preferably about 100 kDa.

The polydispersion index of the LMWHA obtained according to the process of the invention, calculated as weight average mean molar mass (MW_{w}) divided for number average molar mass (MWₙ), is less than 4, preferably less than 2.5.

According to an embodiment of the invention, the process for the preparation of the LMWHA is also characterized by the fact of avoiding the presence of chains with very low molecular weight below 20 kDa in the final product. Said chains having very low molecular weight are indeed known to have proinflammatory activity.

The process of the invention therefore allows to obtain a LMWHA with a specific mean molecular weight in a reproducible way.

Further subject-matter of the present invention is represented by the LMWHA obtained according to the method of the invention.

A further subject-matter of the invention is a LMWHA, obtained according to the method of the invention which shows the following characteristics:
mean molecular weight from 90 kDa and 120 kDa, preferably from 95 kDa to 110 kDa, more preferably about 100 kDa;
linear chains (not crosslinked);
less than 5% of the total chains with a molecular weight lower than 20 kDa, measured by HPLC in the conditions indicated in the experimental section below;
polydispersion index less than 4, preferably less than 3, more preferably less or about 2.5.

The LMWHA of the invention can be used for example in ophthalmic compositions, in which it can be contained in high amount, for example from 0.5 to 2 %, preferably from 1% to 2%, more preferably from 1.1 to 2 %, advantageously from 1.5 to 2% by weight on the volume of the composition.

Such high concentrations of hyaluronic acid in a liquid composition for ophthalmic use can be obtained only thanks to the characteristic molecular weight of the LMWHA of the invention, which, as indicated, falls in a very restricted range. The presence of chains with higher molecular weight in fact would result in an excessive increase of the viscosity, making these compositions unusable. The presence of lower molecular weight chains would lead to toxicity problems due to their pro-inflammatory activity. These compositions can be used as moisturiser and/or as artificial tears and/or to treat the dry eye syndrome.

The ophthalmic composition here described are not a subject-matter of the invention. The invention will be described now in the following experimental section in a illustrating, but not limiting way.

### Experimental Section

Even where not indicated the viscosity was measured by a rotational viscometer Brookfield (DV-II Pro equipped with a small sample adapter and spindle SC-18 at 20° C. and 0.1 rpm.

Even where not indicated, the HPLC was carried out in the following conditions: isocratic system in 0.15 M NaCl buffer pH 7.0, TSK 6000 column with guard-column, run time 30 minutes with 0.5 ml/min flow and UV detector at 205 nm.

### Example 1

5 g of HMWHA, mean molecular weight 1.0 MDa, were dissolved in 250 ml of water at room temperature under stirring. The solution was brought to 60° C., then 250 ml of 0.2 N HCl (pH 1) were added and the solution was kept under stirring. When the solution reached 60° C. the first sample was collected as time zero sample (t0). Other samples were collected at given time intervals, as described in Table 1, and the measure of the viscosity of the solution and mean molecular weight of the hyaluronic acid were performed. The measure of the viscosities was performed by a rotational viscometer (Brookfield DVII-Pro) equipped with a small sample adapter with the spindle SC4-18 at 20° C. at 0.1 rpm, taking 16.5 g of the solution and bringing it to 25 ml of 0.25 M phosphate buffer (pH 8). The molecular weight of the polymer in solution was measured by HPLC in isocratic system with 0.15 M NaCl buffer pH 7, TSK 6000 column with guard column, run time of 30 minutes and flux of 0.5 ml/min and UV detector at 205 nm.

TABLE 1

Viscosity data and mean molecular weight of the samples collected at different times during the depolymerization reaction (FIGS. 1-3 ).

| Sample | Time (minutes) | Viscosity (mPa·s) | Mean molecular weight (kDa) |
|---|---|---|---|
| t₀ | 0 | 746.8 | 1.000 |
| t₆₀ | 60 | 41.27 | 598.5 |
| t₁₂₀ | 120 | 12.87 | 334.4 |
| t₁₈₀ | 180 | 6.63 | 200.6 |
| t₂₁₀ | 210 | 5.22 | 162.0 |
| t₂₄₀ | 240 | 4.46 | 131.6 |
| t₂₇₀ | 270 | 4.00 | 116.3 |

The reaction was stopped after 270 min (t270); the viscosity of the last sample collected was 4 mPa·s. The sample t270 was filtered by a 10,000 Da membrane (Millipore Prepscale) bringing the solution 10 times more concentrated and making a precipitation of the product with 3 volumes of ethanol. The precipitate was kept at 40° C. for 4 hours. The polymer thus isolated showed a mean molecular weight measured by HPLC of 116 kDa, and a distribution index of 1.63, calculated as weight average molecular weight (Mw) divided by the number average molecular weight (Mn).

### Example 2

39.7 g of HMWHA, mean molecular weight 1.4 MDa, were solubilised in 1,998 litres of water. The starting sample of HA in powder was added stepwise under stirring and the full dissolution was obtained in 6 hours. The solution was kept overnight at room temperature, then the temperature was brought to 60° C. under stirring. When the temperature of 60° C. was reached, 1,998 litres of 0.2 N HCl (pH 1) were added to the solution keeping the temperature at 60° C. and stirring. Samples of 16.5 g of solution were collected at different times and diluted to 25 ml with 0.25 M phosphate buffer (pH 8). The viscosity was measured by a rotational viscometer (Brookfield DVII-Pro) equipped with a small sample adapter with the spindle SC4-18 at 20° C. and 0.1 rpm. After 180 minutes of reaction, the sample collected had a viscosity of 4.6 mPa·s. The reaction was stopped by neutralisation with 1 N NaOH (pH 7). The product was filtered by a 10,000 Da membrane (Millipore Prepscale) to concentrate the solution 10 times and then precipitated with 3 volumes of ethanol. The precipitate was dried at 40° C. for 4 hours. The polymer isolated showed a mean molecular weight measured by HPLC of 95 kDa, and a distribution index of 2.55, calculated as weight average molecular weight (Mw) divided by the number average molecular weight (Mn).

### Example 3

60 g of HMWHA, mean molecular weight 1.4 MDa, were solubilised in 3 litres of water. The starting sample of HA in powder was added stepwise at 40° C. under stirring and the full dissolution was obtained in 6 hours. The solution was kept overnight at room temperature, then the temperature was brought to 60° C. under stirring. When the temperature of 60° C. was reached, 3 litres of 0.2 N HCl (pH 1) were added to the solution keeping the temperature at 60° C. under stirring.

Samples of 16.5 g of solution were collected at different times and diluted to 25 ml with 0.25 M phosphate buffer (pH 8). The viscosity was measured by a rotational viscometer (Brookfield DVII-Pro) equipped with a small sample adapter with the spindle SC4-18 at 20° C. and 0.1 rpm.

After 140 minutes of reaction, the sample collected had a viscosity of 4.32 mPa·s. The reaction was stopped by neutralisation with 1 N NaOH (pH 7). The product was filtered by a 10,000 Da membrane (Millipore Prepscale) to concentrate the solution 10 times and precipitating the product with 3 volumes of acetone and washed with isopropanol. The precipitate was dried at 40° C. for 4 hours. The polymer isolated showed a mean molecular weight measured by HPLC of 120 kDa, and a distribution index of 2.45, calculated as weight average molecular weight (Mw) divided by the number average molecular weight (Mn).

### Example 4 - Measure of the Viscosity of LMWHA Containing Solutions

The product obtained according to the depolymerisation reaction described in Example 1, after drying, is dissolved in water at different concentrations.

The viscosity of the solutions is measured by a rotational viscometer Brookfield (DVII Pro) equipped with a small sample adapter and a spindle SC4-18 at 20° C. and 0.1 rpm. The results obtained are reported in Table 2.

TABLE 2

Viscosity of the aqueous solutions at increasing concentration of LMWHA with chains mean molecular weight of 100 kDa.

| concentration (% weight/volume) | viscosity (mPa·s) |
|---|---|
| 4 | 240 |
| 6 | 750 |
| 8 | 2,700 |
| 10 | 6,700 |

### Example 5

5 gr of HMWHA with a molecular weight of 1.4 MDa were dissolved under stirring in 500 ml of water at room temperature. The solution was brought to 60° C. and then 500 ml of 0.8 N HCl were added under stirring and maintained at 60° C. and the viscosity was measured after neutralisation with 1M NaOH. A solution with viscosity of 1.6 mPa.s was obtained after 300 minutes and the molecular weight of the LMWHA was 52,000 Da.

### Example 6

1 gr of the sample isolated in example 1 with mean molecular weight of 116 kDa was dissolved in 50 ml of water at room temperature. The solution was brought to 60° C. Then 50 ml of 0.2N HCl were added and the solution brought to 60° C. under stirring. The viscosity of the samples collected and neutralized with 1M NaOH was measured. The results are shown in Table 3.

TABLE 3

| Sample | Time (minutes) | Viscosity mPa.s | Molecular weight (kDa) |
|---|---|---|---|
| T₆₀ | 60 | 3.2 | 95.3 |
| T₁₂₀ | 120 | 2.84 | 78.5 |
| T₁₈₀ | 180 | 2.54 | 69 |
| T₂₀₀ | 200 | 1.83 | 52 |

### Example 7

50 g of HMWHA with an average molecular weight of 1.3 MDa were dissolved portionwise in 2.5 1 of water at 40° C. under stirring. After one night in water, the solution was brought to 60° C. and 2.5 1 of 0.2 N HCl were added under stirring. The reaction was brought to 60° C. and the viscosity was checked as described in Example 1. After 210 minutes the viscosity measured 5.2 mPa s. so it was stopped by neutralization with 3N NaOH. The average molecular weight of the product obtained after purification by ultrafiltration and precipitation with ethanol measured with HPLC was 105 kDa and the polydispersion 2.8.

### Example 8

20 g of HMWHA with an average molecular weight of 1 MDa were dissolved portionwise in 1 liter of water at room temperature under stirring. After one night at room temperature the solution was heated to 60° C. and 11 of 0.2N HCl was added. The reaction kept at 60° C. and after 210 minutes the viscosity was 5.5 mPa · s. The solution was neutralized with 3N NaOH and the sample recovered by ultrafiltration on a 10,000 Da membrane and precipitated with ethanol. The average molecular weight measured in HPLC was 92 kDa and the polydispersion was 1.85.

### Example 9 - Preparation of a 2% (w/v) Ophthalmic Composition_in LMWHA (not according to the invention)

1.5 g of LMWHA, mean molecular weight (mw) 100 kDa, prepared as described in example 1, were dissolved in 75 ml of 0.08 m phosphate buffer (pH 7.5) under stirring, obtaining a 2% solution of the polymer. The viscosity was measured by a rotational viscometer Brookfield (DVII Pro) equipped with a small sample adapter and a spindle SC4-18 at 20° C. and 0.1 rpm. The dynamic viscosity of the 2% solution of LMWHA with mean molecular weight of 100 kDa was 27.59 mPa.s.

### Example 10 - Preparation of a 1.5% (w/v) Ophthalmic Composition_in LMWHA (not according to the invention)

25 ml of the solution obtained in Example 5 were diluted with 8.3 ml of 0.08 M borate buffer (pH 7.5) under stirring, obtaining a 1.5% solution of the polymer. The viscosity was measured by a rotational viscometer Brookfield (DVII Pro) equipped with small sample adapter and a spindle SC4-18 at 20° C. and 0.1 rpm. The dynamic viscosity of the 1.5% solution of LMWHA with mean molecular weight of 100 kDa was16.80 mPa.s. Said solution was filtered with a 0.22 µm filter and filled in a multidose device suitable for ophthalmic administrations.

### Example 11 - Preparation of a 1% (w/v) Ophthalmic Composition_in LMWHA (not according to the invention)

25 ml of the solution obtained in Example 5 were diluted with 25 ml of 0.08 M borate buffer (pH 7.5) under stirring, obtaining a 1% solution of the polymer. The viscosity was measured by a rotational viscometer Brookfield (DVII Pro) equipped with small sample adapter and a spindle SC4-18 at 20° C. and 0.1 rpm. The dynamic viscosity of the 1% solution of LMWHA with mean molecular weight of 100 kDa was 7.80 mPa.s. This solution was filtered by a 0.22 µm filter and filled in a multidose device suitable for ophthalmic administrations.

1. A process for preparing linear low molecular weight hyaluronic acid (LMWHA), which shows restricted ranges of molecular weight within the range from 50 kDa to 230 kDa, said process comprising
depolymerizing linear high molecular weight hyaluronic acid (HMWHA) in an aqueous medium at pH lower than 2, at a concentration from 0.5 to 1% (w/v), at a temperature from 40 to 80° C.

2. The process according to claim 1, wherein said aqueous medium at pH lower than 2 is made acidic by the addition of a strong, non-oxidizing acid.

3. The process according to claim 1, further comprising:
(i) experimentally predetermining a curve correlating the viscosity of the reaction medium and the consequent molecular weight of the hyaluronic acid therein contained;
(ii) dissolving linear HMWHA in water, at a concentration of about 1-2% (w/v):
(iii) heating the solution and add a strong acid to obtain pH 1-1,5;
(iv) measuring the viscosity of the solution at different times and stopping the depolymerisation by neutralizing with a strong base till pH about 7 once reached the viscosity related to the LMWHA with the desired molecular weight according to the curve of step (i); and
(v) isolating the LMWHA obtained.

4. The process according to claim 1, wherin said acid is hydrochloric acid.

5. The process according to claim 4, wherein said hydrochloric acid is added at a concentration of 0.5-0.3N.

6. The process according to claim 1, wherein said pH is from 1 to 1.5.

7. The process according to claim 1, wherein the depolymerizing step is stopped by addition of a solution of a base, to pH about 7.

8. The process according to claim 7, wherein said base is NaOH.

9. Low molecular weight hyaluronic acid (LMWHA), obtained according to the method of claim 1 and characterized by:
mean molecular weight ranging:
from 50 kDa to 230 kDa; or
from 90 kDa to 120 kDa; or
from 180 kDa to 230 kDa; or
from 190 kDa to 215 kDa; or
from 45 kDa to 60 kDa; or
from 47 kDa to 55 kDa;
uncrosslinked linear chains;
less than 5% of the total chains with a molecular weight lower than 20 kDa, measured by HPLC;
polydispersion index lower than 4.

10. The process according to claim 1, for the preparation of a linear LMWHA with a molecular weight ranging from 90 kDa to 120 kDa, which comprises:
a) dissolving the linear HMWHA in water at a concentration of about 1-2 %(w/v);
b) heating the solution and to add a strong acid to a pH of 1-1.5;
c) measuring the viscosity of the solution at time ranges;
d) stopping the depolymerisation adding a strong base when the viscosity reaches 4-5 mPa.s;
e) isolating the LMWHA so obtained.

11. The process according to claim 10, wherein in step b) said solution is heated at a temperature of 40-80° C.

12. The process according to claim 10, wherein, said acid is hydrochloric acid.

13. The process according to claim 12, wherein said hydrochloric acid is added at a concentration of 0.1-0.3N.

14. The process according to claim 1, wherein said pH is about 1.

15. The LMWHA, obtained according to claim 1 showing the following features
mean molecular weight ranging from 90 kDa to 120 kDa;
linear chains (not crosslinked);
less than 5% of the total chains with a molecular weight lower than 20 kDa;
polydispersion index lower than 4.

16. The LMWHA according to claim 15, wherein the weight average molecular weight is from 95kDa to 110 kDa.

17. The LMWHA according to claim 16, wherein the weight average molecular weight is about 100 kDa.

18. The LMWHA according to claim 9, wherein the molecular weight is about 100 kDa.

19. The LMWHA according to claim 9, wherein the molecular weight is about 200 kDa.

20. The LMWHA according to claim 9, wherein the molecular weight is about 50 kDa.
